(19) United States
(12) Patent Application Publication
(10) Pub. No.: US 2018/0228662 A1
(43) Pub. Date: Aug. 16, 2018

(54) Multi-Functional Personal hygiene Swab

(71) Applicant: Nadia Glasner, Baxter, TN (US)

(72) Inventor: Nadia Glasner, Baxter, TN (US)

(21) Appl. No.: 15/435,238

(22) Filed: Feb. 16, 2017

(51) Int. Cl. A61F 13/38; A45D 40/28; A45D 44/00

(52) U.S. Cl. CPC A61F 13/38; A45D 44/00; A45D 40/28

(57) ABSTRACT

A multi-functional swab for cleaning waste from an ear. The swab includes a first swab tip having a swab pad in a looped shape with an open center and a second swab tip having a unified, whole swab pad, the first and second swab tips being connected by a stem. The swab tips may be constructed of cotton and can be applied around the ends of the stem. Alternatively, the looped swab tip may be made of plastic or possible certain soft metals. In addition, the swab tips may be formed by simply creating a swab pad, e.g. formed of interwoven cotton, and, in the case of the looped swab tip, cutting out a piece of the center. The swab tip with a looped shape may be used to clean the ear but because of the looped structure would be better capable of manipulating and removing waste.

### TECHNICAL FIELD

The present invention relates to the field of personal hygiene swabs. More specifically, the invention relates to a multi-functional personal hygiene swab for cleaning the ear.

### BACKGROUND

Traditionally, swab tips for use of personal hygiene typically have absorbent pads on each end of a stem for cleaning hard to reach areas, e.g. for cleaning within the deep recesses of the ear drum. Although, the swab tips that are currently available on the market perform the base functions needed to clean these hard to reach areas, it does not mean existing swab tips could not be improved to further enhance its functionality so that they may be better suited or adapted to clean even better. For example, because the traditional swab tips are simply absorbent pads, the functional capability may only be to absorb and clean but not perform other useful functions, e.g. encompassing or otherwise capable of manipulating, pulling and extracting waste, that may contribute to the better maintenance of hygiene of the individual and, therefore, health of the individual.

As such, there is a need for a multi-functional swab that is capable of more than simply absorbing waste but also functionally capable of manipulating and maneuvering the waste for cleaning purposes.

### SUMMARY

The example embodiments presented herein meet the above-identified needs by providing a multi-functional personal hygiene swab that includes a first swab tip having a swab pad in a looped shape with a open center and a second swab tip having a singular, unified swab pad, the first and second swab tips being connected by a stem. The stem may be made of plastics or paper products and the swab tips may be constructed of cotton or other like synthetic materials. Alternatively, the swab tip with the looped shape with the open center may be constructed of plastics.

In another embodiment, the personal hygiene swab may include a first swab tip having a looped shape with an open center, a second swab tip with an open center, and a stem connecting the first and second swab. The stem may be made of plastics or paper products and the swabs may be constructed of cotton or other like synthetic materials.

### BRIEF DESCRIPTION OF THE DRAWINGS

FIG. 1 is a perspective view of multi-functional personal hygiene swab according to an embodiment of the invention.

FIG. 2 is a personal hygiene swab according to an embodiment of the invention.

### DETAILED DESCRIPTION

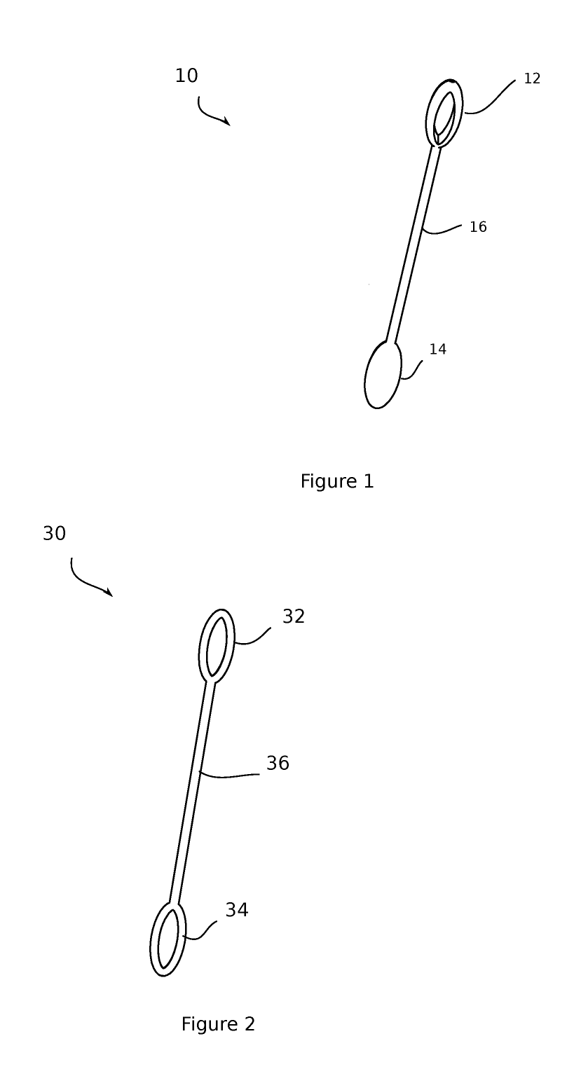

Referring to FIG. 1, illustrated is a perspective view of a multi-functional hygiene swab according to an embodiment of the invention and is denoted generally as 10. The swab 10 includes a first swab tip 12 having a swab pad in a looped shape with an open center and a second swab tip 14 having a singular swab pad, the first and second swab tips being connected by a stem 16. The stem 16 may be made of plastics or paper products and the swabs may be constructed of cotton or other like synthetic materials that can be applied around the ends of the stem. Alternatively, the swab tip 12 may be made of harder, denser material, such as plastic and possible certain soft metals. In addition, the swab tips 12,14 may be formed by simply creating a swab pad, e.g. formed of interwoven cotton, and, in the case of swab tip 12, cutting out a piece of the center. The swap tips 12,14 may be fixed to the ends using an adhesive material, wrapped around the ends, or alternatively, in the case of swab tip 12, formed as an integral part of the stem 16, e.g. using plastic injection molding processes. The swab tip 14 may be used for traditional purposes, such as cleaning of the ear and applying makeup. The swab tip 12 may be used to clean the ear but because of the looped structure is better capable of manipulating and removing waste.

Referring now to FIG. 2, illustrated is a personal hygiene swab according to an embodiment of the invention and is denoted generally as 30. In this embodiment, the hygiene swab 30 may include a first swab tip 32 having a looped shape with an open center, a second swab tip 34 having a looped shape with an open center, and a stem 36 connecting the first and second swab. Alternatively, the swab tip 32,34 may be made of harder, denser material, such as plastic and possible certain soft metals. In addition, the swab tips 32,34 may be formed by simply creating a swab pad, e.g. formed of interwoven cotton, and cutting out a piece of the center. The stem 36 may be made of plastics or paper products and the swabs may be constructed of cotton or other like synthetic materials. The swab 30 in this case is used to clean the ear in a manner that is better capable of manipulating and removing waste.

Thus, While there have been shown and described and pointed out fundamental novel features of the invention as applied to a preferred embodiment thereof, it will be under stood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same Way to achieve the same results are within the scope of the invention. Substitutions of elements from one described embodiment to another are also fully intended and contemplated. It is also to be understood that the drawings are not necessarily drawn to scale but that they are merely conceptual in nature. It is the intention, therefore, to be limited only as indicated by the scope of the claims appended hereto.

1. A multi-functional personal hygiene swab for cleaning waste from an ear, the hygiene swab comprises:
a first swab tip having a swab pad in a looped shape with an open center;
a second swab tip having a singular swab pad; and
a stem having the first swab tip fixed to an end of the stem and the second swab tip fixed to an opposite end of the stem.

2. The multi-functional personal hygiene swab of claim 1 wherein the stem is made of plastic.

3. The multi-functional personal hygiene swab of claim 1 wherein the stem is made of paper.

4. The multi-functional personal hygiene swab of claim 1 wherein the swab tips may be made of plastic.

5. The multi-functional personal hygiene swab of claim 1 wherein the first swab tip may be made of plastic.
